(19) United States
(12) Patent Application Publication
(10) Pub. No.: US 2025/0387521 A1
(43) Pub. Date: Dec. 25, 2025

(54) THERANOSTIC FOR TREATING CANCER

(71) Applicant: CASE WESTERN RESERVE UNIVERSITY, Cleveland, OH (US)

(72) Inventors: James P. Basilion, Shaker Heights, OH (US); Ethan Walker, Willoughby, OH (US); Brian Straight, Charlotte, NC (US)

(21) Appl. No.: 19/191,718

(22) Filed: Apr. 28, 2025

Related U.S. Application Data
(60) Provisional application No. 63/638,990, filed on Apr. 26, 2024.

(51) Int. Cl. A61K 49/00; A61K 41/00; A61P 35/00; C07K 16/28

(52) U.S. Cl. CPC A61K 49/0034; A61K 41/0057; A61K 49/0056; A61P 35/00; C07K 16/2818; C07K 16/2827; A61K 2121/00; A61K 2123/00

(57) ABSTRACT

A theranostic for treating cancer in a subject in need thereof includes a cancer cell-targeted cyanine near-infrared fluorophore for use in a method of (i) fluorescence image guided surgery (FIGS) and (ii) a photodynamic therapy (PDT) and/or a photothermal therapy (PTT).

### RELATED APPLICATION

This application claims priority from U.S. Provisional Application No. 63/638,990, filed Apr. 26, 2024, the subject matter of which IS incorporated herein by reference in its entirety.

### BACKGROUND

The best chance for a cure of many solid tumor cancers is complete removal of all cancer tissue from the body of the patient. Cancer surgeons face a perennial problem of how tumor tissue can be removed from a patient if it cannot be seen during the surgical procedure. While the main bulk of a tumor and margins may be relatively easy to define, for many tumors this is not the case. This uncertainty is especially true for breast cancer patients undergoing breast conservation surgery (BCS); nearly 75% of US breast cancer surgeries are currently BCS (cancer.org). Approximately 80% of BCS surgeries are for invasive cancer. The challenge for BCS is determining where tumor tissue ends, and normal tissue begins. This ability is critical because breast cancer is the most common cancer in women and failure to achieve complete resection involves large numbers of patients (297,790 new US cases of breast cancer were projected for 2023, cancer.org). Surgical removal of the primary cancer with or without adjuvant therapy is the standard of care for this disease. The microscopic status of the margins of the excised lumpectomy specimen is the most important risk factor for local recurrence.

For invasive breast cancer, the pathology term positive surgical margin (PSM) indicates that invasive carcinoma, with or without DCIS, is touching a tissue edge of a lumpectomy specimen, i.e., “cancer at ink” (current SSO-ASTRO guidance recommendations and). PSMs are determined pathologically. Following surgery, the excised specimen is typically marked with ink to provide perimeter orientation, cut into 2-3 mm-thick portions, fixed, paraffin embedded and one (or more) 3-5 μm-thick tissue section is cut from each portion for histological analysis. The margins on each section are then microscopically examined by a pathologist and data reported days after surgery. This approach significantly under samples BCS specimen margins. PSMs require re-excision of any residual cancer tissue, which is estimated to occur between 20-60% of the time requiring patients to return from home for further surgery. This approach is often associated with poorer cosmetic results for breast reconstructions and with increased risk for local and distant recurrence of the disease. PSMs are associated with a 2-fold increase in the risk of local recurrence when compared with negative margins.

In 2017, JAMA Surgery reported that, for women who underwent additional breast cancer surgery, the mean 2-year total health care costs increased by $11,621 for patients undergoing a repeated BCS and $26,276 for patients undergoing a subsequent mastectomy. The total patient economic impact of breast cancer is more than $29 billion annually, not including the national economic impact, which is greater. A follow up study published in 2024 indicated a 21% re-excision rate for commercially insured women, which added an additional $21,607 to costs in year 1 of treatment and was associated with a 54% increase in complications (a rate that jumped to 89% for women in Medicare). Institutions are also negatively impacted as their reimbursements are significantly less for repeat procedures.

Failure to achieve complete resection during the initial surgery has led to significant efforts to identify infiltrative cancer at tumor margins. While several technologies have been developed for real-time margin assessment, none have proven robust enough to gain widespread clinical acceptance because each has significant weaknesses including: frozen sectioning and touch prep cytology; Intraoperative radiography of the excised tissues; radiofrequency spectroscopy; optical coherence tomography; radio-impedance detection; and ultrasound visualization.

The weaknesses of current surgical and pathological methods and their failure to meet important, unmet clinical needs, have spurred the development of fluorescent molecular probes that “light up” cancer, making intraoperative assessment of surgical resection possible. There is abundant clinical evidence that targeted near-infrared fluorescent (NIRF) molecular probes can enhance the ability to discriminate between normal and tumor tissue and are safe for both patients and the surgical team. However, local recurrence after fluorescence image-guided surgery (FIGS) has not yet been assessed. Occult disease may likely be “invisible”, i.e., not detectable to intraoperative imaging approaches, potentially leading to local disease recurrence and metastasis.

### SUMMARY

Embodiments described herein relate to a theranostic for treating cancer in a subject in need thereof and particularly to the use of the cancer cell-targeted cyanine near-infrared fluorophore in a method of (i) fluorescence image-guided surgery (FIGS) and (ii) a photodynamic therapy (PDT) and/or a photothermal therapy (PTT) for treating cancer.

In some embodiments, a method for treating cancer using the theranostic includes administering to the subject the cancer cell-targeted cyanine near-infrared fluorophore. The administered cancer cell-targeted cyanine near-infrared fluorophore is detected in the subject to determine the location and/or distribution of the cancer cells in the subject. The cancer cells detected using the administered cancer cell-targeted cyanine near-infrared fluorophore at the determined location and/or distribution in the subject are surgically resected. Remaining or residual cells at the determined location after surgical resection that are detected by, bound to, and/or complexed with the cancer cell-targeted cyanine near-infrared fluorophore are irradiated at a wavelength effective to ablate the remaining cancer cells.

### BRIEF DESCRIPTION OF THE DRAWINGS

FIG. 1 illustrates activation of quenched, substrate-based FIGS probe.

FIGS. 2(A-B) illustrate tumor specific bio-luminescence signals correlate with AKRO-6qc dependent ICG fluorescence in tumor-bearing mice. A. A representative mouse bearing 2 4T1/luciferase orthotopic breast cancers tail vein injected with 2.3-mg/kg of AKRO-6qc and imaged in vivo at 6-h. B. After imaging as in (A) mice were sacrificed, all tumors and organs were excised and imaged. Notes: All tumors and one organ set are presented, N=3. 2) IVIS Spectrum used for imaging and display.

FIG. 3 illustrates accumulation of AKRO-6qc in tumors suggests that 24-36 h after systemic administration may be the optimal time-window for PTT. Time-course of ICG fluorescence as total radiance efficiency from orthotopic tumors. Inset-24 h ICG fluorescence of 4T1 xenografts (blue arrows) of a representative mouse AKRO-6qc dosed at 2.3-mg/kg. Notes: Vertical bars—±SD, n=3.

FIGS. 4(A-B) illustrate AKRO-6qc probe induced a significant temperature increase in human breast cancer cells (MDA-MB-468) in vitro. A. Photothermal images of maximum temperature achieved for 10⁷ cells as pellets (green dotted squares) after 300- or 600-J/cm² dose of light irradiation. Maximum temperature reached in cell pellets in displayed in boxed texts. B. Cell pellet temperature at different time points during irradiation. Percentages in blue report cell viability (measured by trypan blue) at the particular temperature (Magnified Plot Symbols). Notes: 1) Controls: MDA-MB-468 cells alone in medium irradiated with light (no AKRO-6qc); AKRO-6qc: MDA-MB-468 cells incubated for 30-min with probe (10-μM) washed and then irradiated with light; 2) Light source—Modulight-7710 with ICG 800-nm filter; 3) Thermal camera—FLIR; 4) Vertical bars+/−SD; 5) Statistics—Student's t-test.

FIG. 5 illustrates the temperature in the surgical cavities during PTT in vivo. PTT thermal imaging (FLIR) of normal and tumor tissue under light exposure (Modulight) in presence or absence of AKRO-6qc. Green arrows—surgical cavity after partial resection of heterotopic breast cancer xenografts. Black arrows—surgical cavity in normal muscle. Note: Temperature above the coagulative threshold (>60° C.) was found during 3-min PTT only in the surgical cavity after partial tumor resection with presence of AKRO-6qc.

FIG. 6 illustrates efficacy of combined FIGS and PTT in vivo. ICG fluorescent imaging (FLARE, Curadel) before and after FIGS followed by PTT. Yellow arrows—breast cancer heterotopic tumor xenograft. Red dotted circle—surgical cavity after FIGS+PTT showing total ablation of the tumor.

FIGS. 7(A-C) illustrate combination of FIGS and PTT extended animal survival and delayed tumor recurrence and metastasis. A. Balb/c nude mice were heterotopically implanted with 4T1-Luc tumors. At the appropriate time tumors underwent surgical resection. Tumor recurrence after WLS, FIGS, and FIGS+PTT was measured using bioluminescence (BLI). Insert—BLI showing remaining tumor cells after complete WLS removal of primary tumor; B. Kaplan-Meier survival curves; and C. Appearance of breast cancer metastasis in lungs of mice from experimental groups measured using BLI. Insert-Representative BLI images of supine mice showing metastatic disease in all groups except those undergoing FIGS+PTT. From top to bottom: Control; WLS; FIGS; FIGS+PTT. Notes: Black arrows—day of surgery of sham WLS (controls) and WLS; Red arrows—day of surgery for FIGS and FIGS+PTT. Statistics: *-p=0.05, FIGS+PTT vs. WLS; **-p=0.05, FIGS+PTT vs. sham WLS (controls), WLS, and FIGS; #-p=0.01, FIGS+PTT vs. sham WLS (controls), WLS, and FIGS. Data obtained using IVIS Spectrum.

FIG. 8(A-C) illustrates macrophages uptake and activate AKRO-6qc. A, Tumor tissue macrophages (Mφ) accumulate and activate AKRO-6qc and are most dense at peripheral zone of the tumor xenograft. Overlay of F4/80 Mφs (red) and AKRO-6qc fluorescence (blue) reveals pink overlays showing macrophages take up AKRO-6qc. Notes: 1) Dotted grey lines—edge of the tissue sample; 2) White arrows—location of Mφ which are impregnated with AKRO-6qc. B. Type 2 Mφ (Arg1+, F4/80+) overlaid unto AKRO-6qc, indicated by white dotted circles, suggest that M2 Mφ make up a large proportion of the cells that activate AKRO-6qc. C. High magnification demonstrates that many M1 Mφ do not activate AKRO-6qc. Notes: 1) Frozen sections (12-μm) of the breast tumor mass were prepared and stained for Mφ 2-hr after treatment with AKRO-6qc (ICG) in vivo; 2) Grey arrows—Type-1 Mφ, white arrows—Type-2 Mφ.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The terms “polynucleotide sequence” and “nucleotide sequence” are also used interchangeably herein.

“Recombinant,” as used herein, means that a protein is derived from a prokaryotic or eukaryotic expression system.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “wild type” refers to the naturally-occurring polynucleotide sequence encoding a protein, or a portion thereof, or protein sequence, or portion thereof, respectively, as it normally exists in vivo.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to a theranostic for treating cancer in a subject in need thereof and particularly to the use of a cancer cell-targeted cyanine near-infrared fluorophore in a method of (i) fluorescence image-guided surgery (FIGS) and (ii) a photodynamic therapy (PDT) and/or a photothermal therapy (PTT) for treating cancer.

Surgical removal of solid tumors is often the first step in cancer treatment. Much effort has been spent recently in developing surgical tools that allow better detection of tumor margins and identification of invasion and metastasis. Recent advances in this area include the development of molecularly targeted fluorescent imaging agents that aid the surgeon in accurately distinguishing normal from neoplastic tissue in real time. Of particular importance is the use of Near-Infrared (NIR) fluorophores which provide greater depth of light penetration and are detected at wavelengths where autofluorescence or interference from hemoglobin and other endogenous components is minimal.

In some embodiments, a method for treating cancer using the theranostic includes administering to the subject a cancer cell-targeted cyanine near-infrared fluorophore. The administered cancer cell-targeted cyanine near-infrared fluorophore is detected in the subject to determine the location and/or distribution of the cancer cells in the subject. The cancer cells detected using the administered cancer cell-targeted cyanine near-infrared fluorophore at the determined location and/or distribution in the subject are surgically resected. Remaining or residual cells at the determined location after surgical resection that are detected by, bound to, and/or complexed with the cancer cell-targeted cyanine near-infrared fluorophore are irradiated at a wavelength effective to ablate the remaining cancer cells.

In some embodiments, the cancer cell-targeted cyanine near-infrared fluorophore is a cancer cell-targeted heptamethine cyanine near-infrared fluorophore.

In some embodiments, the cyanine near-infrared fluorophore includes indocyanine green (ICG) or an analogue thereof.

The cyanine near-infrared fluorophore is linked to at least one cancer cell targeting moiety. The cancer cell targeting moiety can bind to, complex with, and/or be cleaved by a cancer cell surface molecule and/or a molecule in a microenvironment of the cancer cell.

In some embodiments, the cancer cell surface molecule and/or molecule in the cancer cell microenvironment includes at least one of cancer cell surface antigen and/or enzyme that is overexpressed in the cancer cell microenvironment.

In some embodiments, the cancer cell antigen includes at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cathepsin, cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

In some embodiments, the targeting moiety includes at least one a peptide, protein, nucleic acid, or small molecule that targets the cancer cell antigen.

In some embodiments, cell-targeted heptamethine cyanine near-infrared fluorophore has the formula:

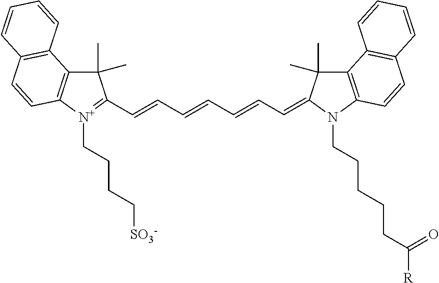

or a pharmaceutically acceptable salt thereof;
wherein R includes the targeting moiety and optional spacer.

In other embodiments, the cancer cell surface molecule and/or a molecule in a microenvironment of the cancer cell is an enzyme that is overexpressed in the cancer cell microenvironment, such as cathepsin.

In some embodiments, the cell-targeted heptamethine cyanine near-infrared fluorophore comprises a cathepsin cleavable heptamethine cyanine near-infrared fluorophore, such as AKRO-6qc (6qc-ICG) or VGT-309.

AKRO-6qc has the following structure:

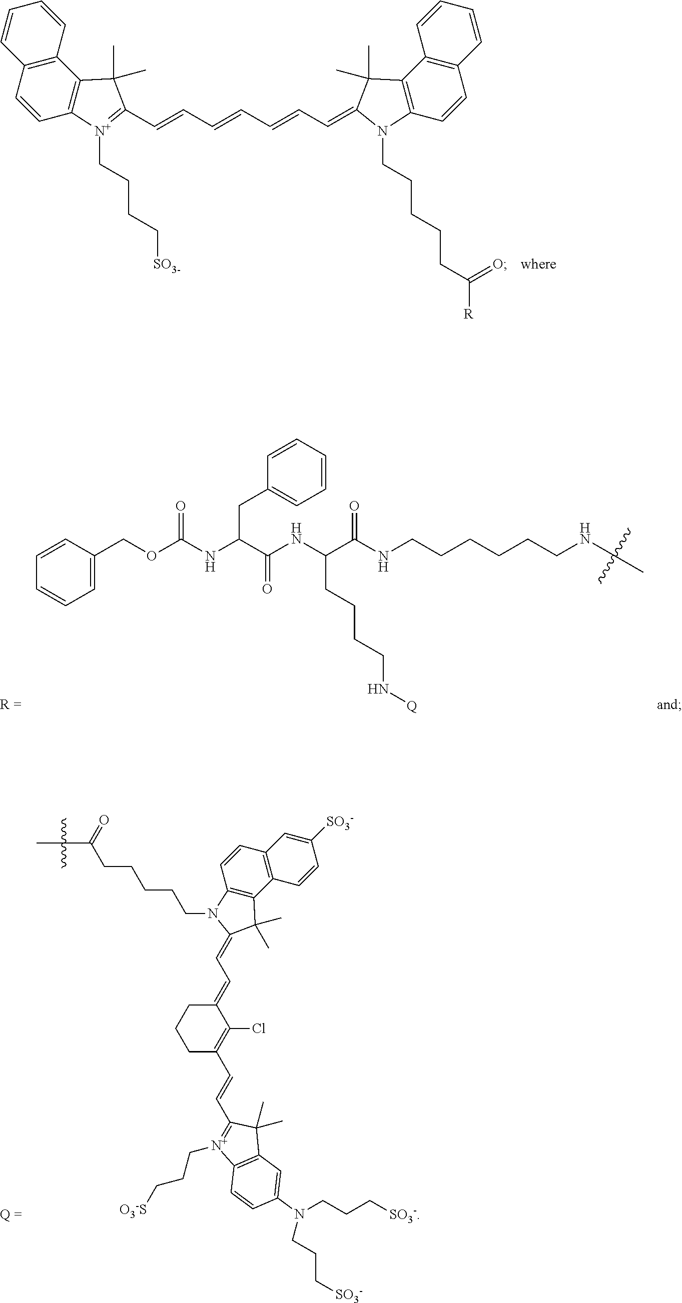

VGT-309 has the following structure:

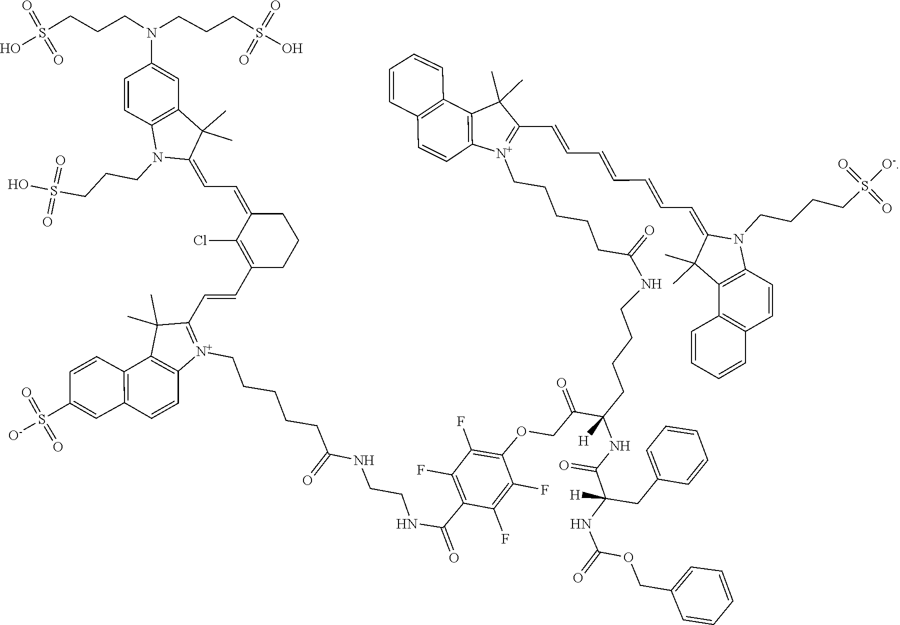

In some embodiments, the cancers detected and/or treated by the cancer cell-targeted cyanine near-infrared fluorophore described herein can include colorectal cancer, breast cancer, lung cancer, melanoma, hepatoma, head and neck cancers, glioma, squamous cell carcinomas of the lung, ovarian cancer, uterine cancer, prostate cancer, gastric carcinoma, cervical cancer, esophageal carcinoma, bladder cancer, prostate cancer, kidney cancer, brain cancer, bone cancer, pancreatic cancer, skin cancer, cutaneous or intraocular malignant melanoma, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemias solid tumors of childhood, lymphocytic lymphoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, medulloblastoma pilomatrixomas, endometrial cancer, multiple myeloma, or T-cell lymphoma.

In other embodiments, the cancer cells can include at least one of a glioma, lung cancer, melanoma, breast cancer, or prostate cancer cell.

In some embodiments, the cancer cell-targeted cyanine near-infrared fluorophore can be administered to the subject by, for example, systemic, topical, and/or parenteral methods of administration. These methods include, e.g., injection, infusion, deposition, implantation, or topical administration, or any other method of administration where access to the tissue by the near-infrared imaging agent is desired. In one example, administration of the cell-targeted cyanine near-infrared fluorophore can be by intravenous injection of the cancer cell-targeted cyanine near-infrared fluorophore in the subject. Single or multiple administrations of the cancer cell-targeted cyanine near-infrared fluorophore can be given. “Administered”, as used herein, means provision or delivery of the cancer cell-targeted cyanine near-infrared fluorophore in an amount(s) and for a period of time(s) effective to label cancer cells in the subject.

Cancer cell-targeted cyanine near-infrared fluorophores described herein can be administered to a subject in a detectable quantity of a pharmaceutical composition containing the cancer cell-targeted cyanine near-infrared fluorophore or a pharmaceutically acceptable water-soluble salt thereof, to a patient.

Formulation of the cancer cell-targeted cyanine near-infrared fluorophore to be administered will vary according to the route of administration selected (e.g., solution, emulsion, capsule, and the like). Suitable pharmaceutically acceptable carriers may contain inert ingredients that do not unduly inhibit the biological activity of the near-infrared imaging agents. The pharmaceutically acceptable carriers should be biocompatible, e.g., non-toxic, non-inflammatory, non-immunogenic, and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A “detectable quantity” means that the amount of the cancer cell-targeted cyanine near-infrared fluorophore that is administered is sufficient to enable detection of binding of the near-infrared imaging agent to the cancer cells. An “imaging effective quantity” means that the amount of the cancer cell-targeted cyanine near-infrared fluorophore that is administered is sufficient to enable fluorescent imaging of binding of the cancer cell-targeted cyanine near-infrared fluorophore to the cancer cells.

The cancer cell-targeted cyanine near-infrared fluorophore administered to a subject can be used to detect and/or determine the presence, location, and/or distribution of cancer cells in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the cancer cell-targeted cyanine near-infrared fluorophore in the animal's tissue, e.g., brain tissue, can be visualized with a near-infrared fluorescence (NIRF) scanner. In one example, the NIRF scanner may be handheld. In another example, the NIRF scanner may be miniaturized and embedded in an apparatus (e.g., micro-machines, scalpel, neurosurgical cell removal device).

“Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the near-infrared imaging agent may then be correlated with the presence or absence of cancer cells in the tissue. A distribution may be dispositive for the presence or absence of cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the near-infrared imaging agent. In this case, the baseline and post-administration images can be compared to ascertain the presence, absence, and/or extent of a particular disease or condition.

In one aspect, the cancer cell-targeted cyanine near-infrared fluorophore may be administered to a subject to assess the distribution of cancer cells in a subject and correlate the distribution to a specific location. Surgeons routinely use intra-operative fluorescent imaging in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination.

Agents described herein that include a cancer cell-targeted cyanine near-infrared fluorophore can be used in intra-operative imaging (IOI) techniques to guide surgical resection and eliminate the “educated guess” of the location of the tumor margin by the surgeon. Previous studies have determined that more extensive surgical resection improves patient survival. Stummer W, Novotny A, Stepp H, Goetz C, Bise K, Reulen H J (2000) Thus, a cancer cell-targeted cyanine near-infrared fluorophore that functions as diagnostic molecular imaging agents have the potential to increase patient survival rates.

In some embodiments, the cancer cell-targeted cyanine near-infrared fluorophore upon administration to the subject can target and detect and/or determine the presence, location, and/or distribution of cancer cells in an organ or body area of a patient. In one example, the cancer cell-targeted cyanine near-infrared fluorophore can be combined with intraoperative imaging (IOI) to identify malignant cells that have infiltrated and/or are beginning to infiltrate at a tumor margin. The method can be performed in real-time during brain or other surgery. The method can include local or systemic application of the cancer cell-targeted cyanine near-infrared fluorophore described herein. A fluorescent imaging modality can then be used to detect and subsequently gather image data. The resultant image data may be used to determine, at least in part, a surgical treatment. Alternatively, this image data may be used to control, at least in part, an automated surgical device (e.g., laser, scalpel, micromachine) or to aid in manual guidance of surgery.

In one example, a cancer cell-targeted cyanine near-infrared fluorophore can be topically applied as needed during surgery to interactively guide a surgeon and/or surgical instrument to the remaining abnormal cells. The cancer cell-targeted cyanine near-infrared fluorophore may be applied locally in low concentration, making it unlikely that pharmacologically relevant concentrations are reached. In one example, excess material may be removed (e.g., washed off) after a period of time (e.g., incubation period).

Following administration and localization of the cancer cell-targeted cyanine near-infrared fluorophore to the targeted cancer cells and surgical resection of the targeted cancer cells, the remaining non-resectable or remaining cancer cells can be exposed to a therapeutic amount of light that causes cancer cell ablation, damage and/or suppression of the remaining cancer cells.

In some embodiments, the cancer cells can be ablated using image-mediated phototherapy. Image-mediated phototherapy can include imaging-guided photothermal therapy (PTT) and imaging-guided photodynamic therapy (PDT). In PTT, the cancer cell-targeted cyanine near-infrared fluorophore bound to the cancer cell or another cell in the cancer cell microenvironment can be irradiated with a wavelength of light effective to convert light energy into heat and ablate the cancer. Advantageously, the as-produced heat can potentially cause thermal expansion of the cancer tissue to generate photoacoustic imaging (PAI) signal. Alternatively, the cancer cell-targeted cyanine near-infrared fluorophore bound to the cancer cell or another cell in the cancer cell microenvironment can be irradiated with wavelength of light effective produce singlet oxygen (O₂) or other reactive oxygen species (ROS) under laser irradiation to induce apoptosis or necrosis of cancer cells, which can be applied for imaging-guided photodynamic therapy (PDT) or further to achieve synergistic PDT/PTT. Only the cells that are exposed simultaneously to the cancer cell-targeted cyanine near-infrared fluorophore and light are destroyed while surrounding healthy, non-targeted and nonirradiated cells are spared from photodamage. Furthermore, the fluorescence of the cancer cell-targeted cyanine near-infrared fluorophore enables simultaneous diagnostic optical imaging that can be used to guide the cancer treatment.

The light, which is capable of activating the cancer cell-targeted cyanine near-infrared fluorophore for PTT and/or PDT agent can be delivered to the targeted cancer cells using, using for example, semiconductor laser, dye laser, optical parametric oscillator or the like. It will be appreciated that any source light can be used as long as the light excites the near-infrared imaging agent.

In some embodiments, the wavelength effective to ablate the remaining cancer cells is a wavelength effective for photodynamic therapy or photothermal therapy.

In some embodiments, the cancer cell-targeted cyanine near-infrared fluorophore described herein can be administered alone as a monotherapy, or in conjunction with or in combination with one or more additional therapeutic agents. For example, the method further includes administering to the subject an immune checkpoint inhibitor.

In some embodiments, the immune checkpoint inhibitor is an inhibitor for any of immune checkpoint molecules selected from the group consisting of PD-1, CTLA-4, TIM-3, BTLA, LAG-3, A2aR, KIR, VISTA, TIGIT, PD-L1 PD-L2, CD80, CD86, GAL-9, HVEM, CD160, MHC class II, B7-H3, B7-H4, B7-H5. B7-H6, and B7-H7, or a combination of two or more inhibitors thereof.

In some embodiments, the immune checkpoint inhibitor is selected from an antibody against the immune checkpoint molecule, an antigen-binding fragment of the antibody, or a combination thereof. For example, the immune checkpoint inhibitor is selected from the group consisting of nivolumab, pembrolizumab, cemiplimab, avelumab, atezolizumab, and durvalumab.

In some embodiments, the cancer cell-targeted cyanine near-infrared fluorophore described herein can be administered to the subject prior to, during, or post administration of an additional therapeutic agent and the distribution of metastatic cells can be targeted with the therapeutic agent. The cancer cell-targeted cyanine near-infrared fluorophore can be administered to the animal as part of a pharmaceutical composition comprising the cancer cell-targeted cyanine near-infrared fluorophore and a pharmaceutically acceptable carrier or excipient and, optionally, one or more additional therapeutic agents. The cancer cell-targeted cyanine near-infrared fluorophore described herein and additional therapeutic agent can be components of separate pharmaceutical compositions, which can be mixed together prior to administration or administered separately. The cancer cell-targeted cyanine near-infrared fluorophore described herein, for example, can be administered in a composition containing the additional therapeutic agent, and thereby, administered contemporaneously with the agent. Alternatively, the cancer cell-targeted cyanine near-infrared fluorophore and therapeutic agent described herein can be administered contemporaneously, without mixing (e.g., by delivery of the agent on the intravenous line by which the therapeutic agent is also administered, or vice versa). In another embodiment, the cancer cell-targeted cyanine near-infrared fluorophore described herein can be administered separately (e.g., not admixed), but within a short time frame (e.g., within 24 hours) of administration of the therapeutic agent.

The cancer cell-targeted cyanine near-infrared fluorophore and therapeutic agent described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of disclosed cancer cell-targeted cyanine near-infrared fluorophore, and therapeutic agent described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the near-infrared imaging agent described herein can also depend on the disease state or condition being treated along with the clinical factors and the route of administration of the near-infrared imaging agent.

The cancer cell-targeted cyanine near-infrared fluorophore described herein can be administered to the subject in conjunction with an acceptable pharmaceutical carrier or diluent as part of a pharmaceutical composition for therapy. Formulation of the cancer cell-targeted cyanine near-infrared fluorophore to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The following example is for the purpose of illustration only and are not intended to limit the scope of the claims, which are appended hereto.

### EXAMPLE

This example describes a theranostic technology to aid in visual identification of the tumor during surgery and subsequent ablation of non-resectable or residual tumor cells not readily identified at surgery. Coupling of cancer-targeted agents with a photodynamic therapy agent (PDT) and/or photothermal therapy agent (PTT) may offer advantages over current treatments. PDT/PTT targeted to tumor cells can increase M2 tumor associated macrophages (TAMs) causing immune suppression and disease recurrence. Current proposed PDT/PTT treatments envision the use of checkpoint inhibitors to offset this effect. The FDA-approved fluorophore Indocyanine Green (ICG) has shown some promise for PTT therapy. ICG has several disadvantages, however, including concentration-dependent aggregation, poor aqueous stability, and lack of target specificity. Research with cancer targeted ICG nanoparticles, while encouraging, is confounded by the complex manufacture of nanoparticles that will likely face significant additional regulatory hurdles. Moreover, the effectiveness of targeting tumor biomarkers may be reduced by tumor heterogeneity of biomarkers.

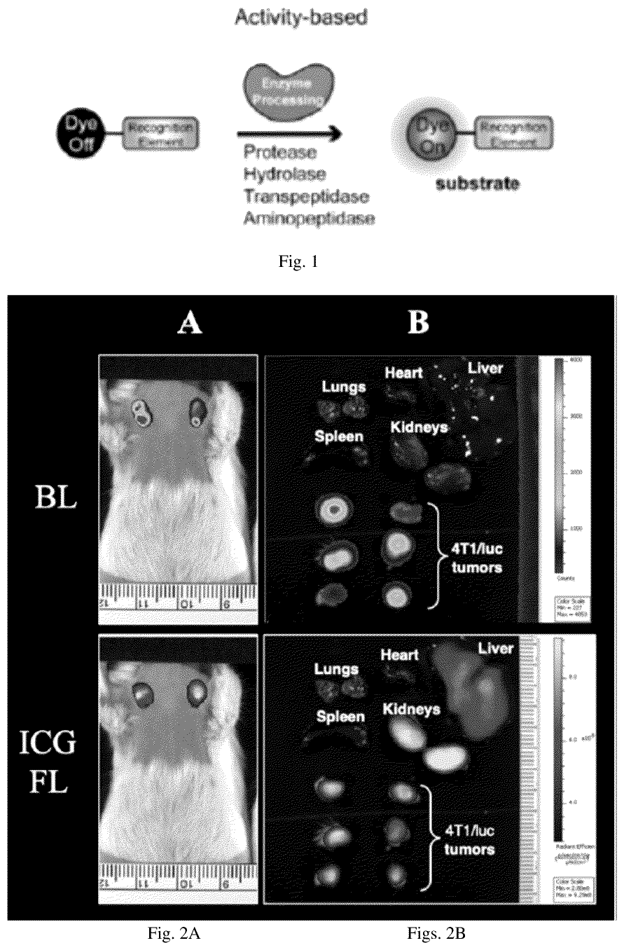

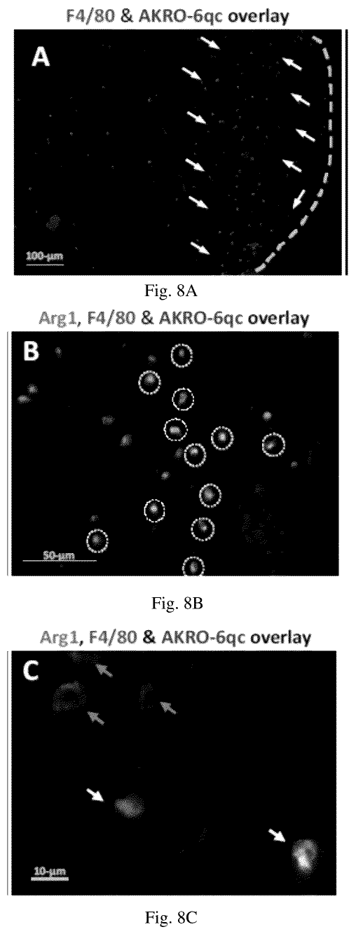

We developed an ICG-based small molecule theranostic technology that enables more complete tumor resection while simultaneously ablating non-detectable microscopic disease. We used Akrotome's existing cathepsin-targeted substrate-based probe technology (AKRO-6qc) to develop its theranostic capabilities. AKRO-6qc is a quenched substrate-based probe technology that utilizes Indocyanine Green (ICG) as the fluorophore and can identify cancers in vivo during surgical procedures. Further, ICG has been documented to have photothermal therapeutic effects suggesting that AKRO-6qc may have potential as a theranostic agent. ARKO-6qc targets cancer-associated and activated cysteine cathepsins that are directly responsible for cancer growth and metastasis, FIG. 1. As these targets are produced both by the tumor as well as by host cells (mostly macrophages) within the tumor in response to cancer, we believe the challenges presented to existing targeted treatments by tumor heterogeneity may also be mitigated. Interestingly, within the tumor almost all M2 macrophages (Arg1+, F4/80+) take up and activate AKRO-6qc (FIG. 8) and PTT may therefore also eliminate M2 macrophages, which suppress the immune response to the tumors. While current adjuvant therapies (radiation and chemotherapy) are effective in preventing tumor recurrence, they are expensive and may have a negative impact on patient quality of life. The coupling of FIGS and PTT may also have the potential to reduce or even eliminate adjuvant therapies after BCS.

Extensive previous research has shown AKRO-6qc is highly selective for its cathepsin targets, exhibiting high sensitivity and specificity. Toxicology studies for AKRO-6qc as a FIGS agent have been successfully completed and the agent is well tolerated by both rodents and canine subjects (see rodent toxicology attachment; dog study ongoing). By studying and optimizing the theranostic capability of AKRO-6qc, we envision future supplementation of the FIGS capability of the probe, which will soon be in clinical trials. We demonstrate herein the potential clinical viability of FIGS/PTT approach by 1) optimizing probe performance for in vivo detection of BCa, and 2) Assessing the performance of the probe by comparing white light, FIGS, and FIGS+PTT resections.

Cancer-associated proteases are among some of the most consistently overexpressed tumor-associated markers. Proteases hydrolyze peptide bonds in proteins and are involved in various physiological processes such as digestion, cell cycle regulation, proteolysis, extracellular matrix remodeling, apoptosis and pro-protein activation. Akrotome's technology has developed quenched protease substrate probes that enter cells. When the probe encounters active proteases, the quencher is hydrolyzed from the substrate and leaves the cell. The resulting charge on the remaining fluorophore “locks” it within the cell, where it accumulates over time, FIG. 1. Several studies have demonstrated the utility of quenched NIRF molecular imaging substrate-based probes for imaging tumor proteases in animal models of cancers, including our own work and recent human trials have highlighted efficacy for quenched activity-based protease probes for FIGS. Of particular interest is that the family of cysteine proteases that robustly activate AKRO-6qc, more specifically cathepsin-B and -L, are highly expressed and active in breast cancer cells and activated M2 macrophages found in the breast cancer tumor microenvironment. Previous studies have demonstrated AKRO-6qc (referred to as 6qc-ICG in the original references) as a FIGS agent showing: AKRO-6qc targets tumor-activated cathepsins with high sensitivity and specificity. Since the probe selectively accumulates in solid tumor cancers that express these cathepsins with a high fluorescent signal to noise ratio the accumulation is likely to be sufficient to exploit ICG from AKRO-6qc as a potential PTT agent. In vivo studies with a precursor probe, using the same quencher and a similar fluorophore, indicated probe uptake almost exclusively by 4T1 tumor-associated macrophages (see FIG. 5). Based upon these published preliminary studies, we believe AKRO-6qc has potential as a PTT agent not only for breast cancer, but also for other cancers which overexpress these proteases.

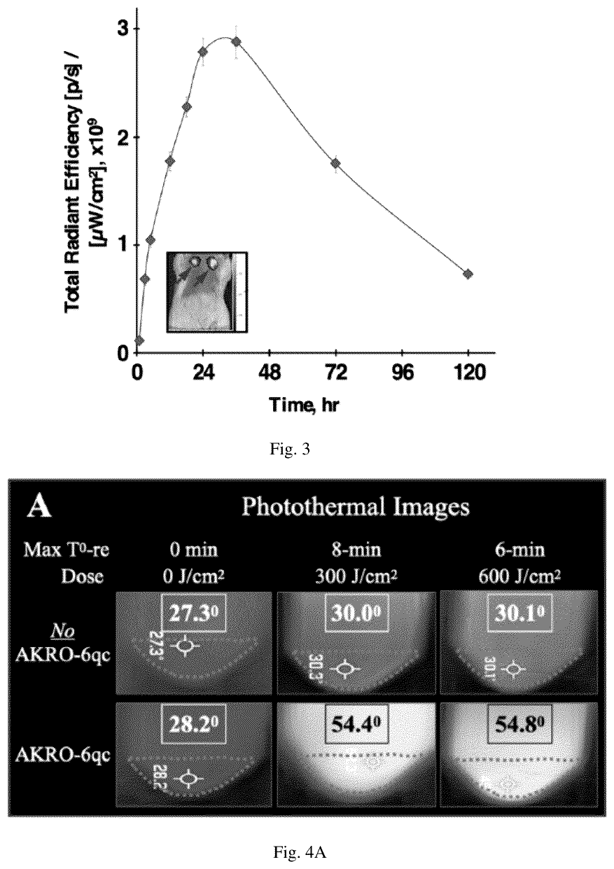

Recently, we performed a series of in vivo studies using MDA-MB468 and 4T1/luc human BCa cells to determine if AKRO-6qc could 1) generate heat after uptake by BCa cells and irradiation with 808 nm light; 2) if irradiation resulted in cell killing; and 3) if FIGS combined with PTT offered benefits in terms of tumor recurrence and survival) over FIGS alone. Our work has established that tumor specific bioluminescence correlates with targeted accumulation of AKRO-6qc in tumor cells (FIG. 2). Furthermore, measurement of ICG fluorescence suggests that the highest levels occur 24-36 h after systemic administration. As achieving optimal PTT temperature is dependent on probe accumulation levels, this time period appears to be also optimal for performing PTT (FIG. 3).

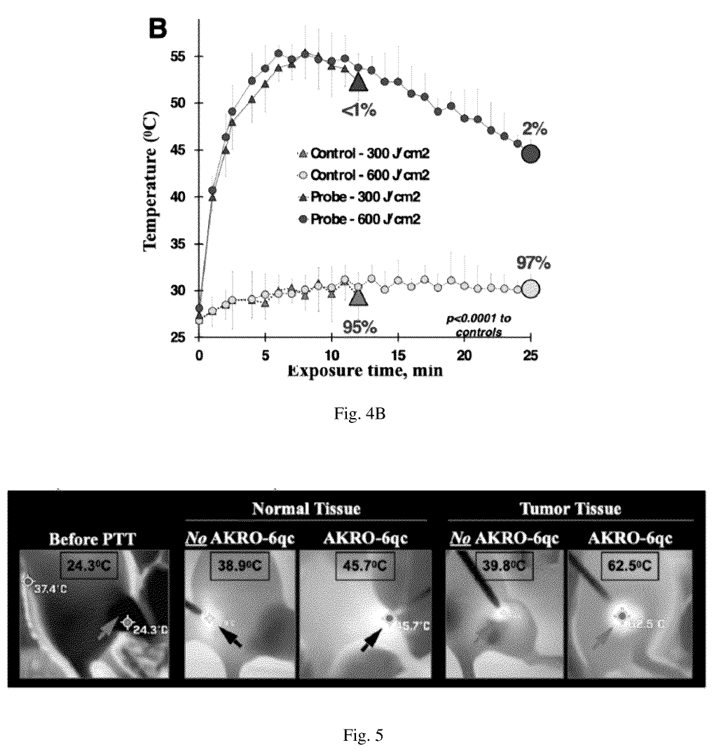

FIG. 4 shows that irradiation only significantly increased temperature in cells pre-incubated with AKRO-6qc with a resultant increase in cell killing of >95% within 12.5 minutes. As ICG is an efficient PTT agent, it is likely that PTT contributed substantially to cellular death. FIG. 5 illustrates this temperature increase in the surgical cavity for residual tumor cells (remaining after partial resection) in which probe has accumulated. FIG. 6 shows the effectiveness of FIGS+PTT in ablation of tumor in the surgical cavity.

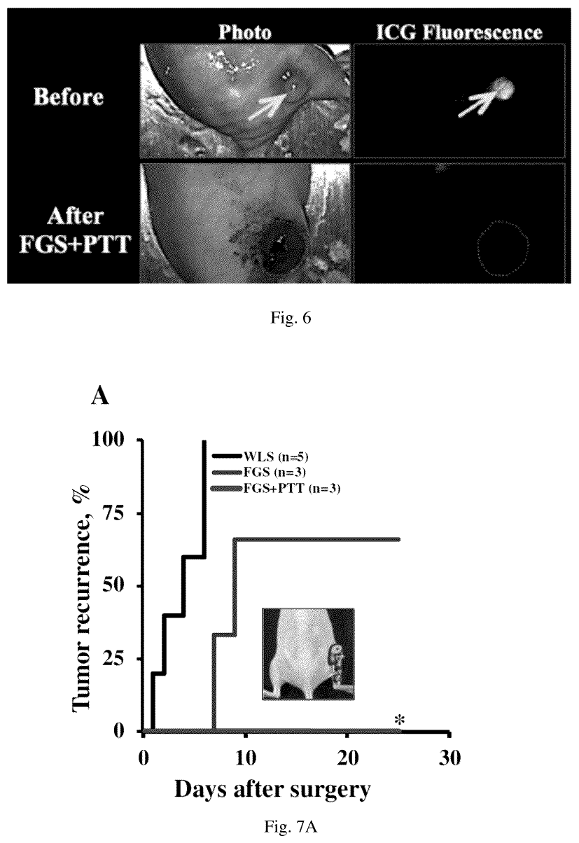

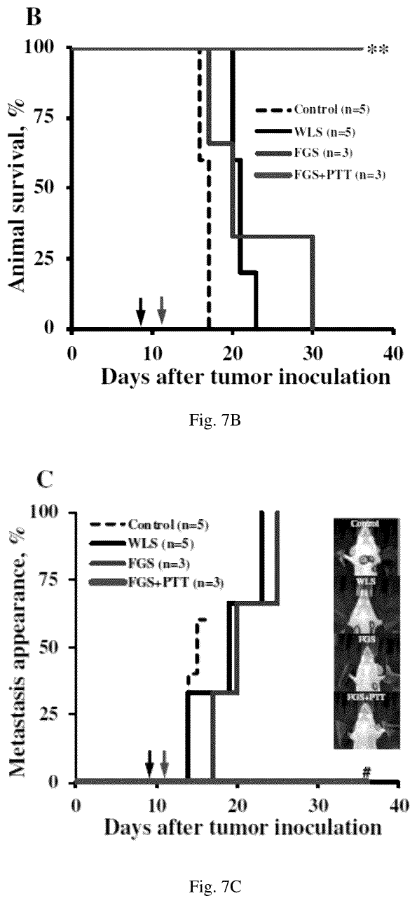

We also performed a study to assess whether the combination of AKRO-6qc-driven FIGS and PTT had a salutary effect on tumor recurrence and metastasis. The study used 4 (Balb/c nu/nu) mice. The animals were implanted with 4T1-luc BCa tumors. After 11 days the mice were divided into four brackets: the control received sham white light surgery (dotted line Figs. B&C); for surgical groups: Group 1, a white light resection was performed; Group 2 received FIGS; Group 3 underwent FIGS resection followed by PTT. FIG. 7 demonstrates the potential of FIGS followed by PTT for the treatment of aggressive breast cancer and, potentially, other solid tumors. Group 3 alone demonstrated both extended survival and absence of lung metastasis for the duration of the experiment (animals were followed to 35 days post tumor inoculation). In contrast when PTT was not administered, groups 1 and 2, significant metastasis to the lungs was detected by Bioluminescence. The complete lack of metastasis to the lungs with treatment (FIGS+PTT) at the primary tumor was remarkable and unexpected, necessitating further investigation.

We will utilize the syngeneic 4T1 tumor model and xenograft human tumors in nude mice to test the hypothesis that: AKRO-6qc will be selectively taken up into cancer cells and TAMs within the mouse tumors and that irradiation with 808 nm light will result in tumor destruction, killing of TAMs, and an increase in the host immune response to 4T1 tumors after PDT/PTT (syngeneic model).

In vivo assays will be conducted to determine the optimal accumulation of probe in mouse BCa. We will perform dosing studies to optimize probe accumulation in the tumors. The half-life of the probe in blood, time to peak accumulation, maximum fluorescent signal will be measured, and biodistribution will be measured. IHC and fluorescence microscopy will be used to identify the location and which tumor/tumor associated cells take up the probe. Metrics: Fluorescent signal of at least 2× over background or greater within 10 minutes of dosage; Statistically significant blood half-life measurements; statistically significant biodistribution data; and biodistribution of AKRO-6qc among tumor cells.

Mice will be randomly divided into three groups: white light surgery (WLS), fluorescence guided surgery (FIGS), and FIGS, followed by PTT (FIGS+PTT). AKRO-6qc will be delivered to all mice followed by surgery. WLS will be performed under room light. For FIGS, tumors will be removed under the guidance of real-time fluorescent camera imaging and the wounds will be sutured for both WLS and FIGS groups. For the FIGS+PDT group, the resection bed will be directly irradiated with 808-nm laser using an optical fiber diffuser under control of a thermal camera and temperatures achieved by irradiation will be monitored using a thermal camera. After PTT the wounds will be sutured.

All three groups will be assessed for tumor ablation, recurrence, and metastasis. Bioluminescent and fluorescent surveys will be conducted for 80 days post-surgery and results per group tabulated. Lungs will also be assessed for the presence of metastatic disease. Survival curves will be plotted. Metrics: AKRO-6qc driven FIGS is superior for tumor resection compared with WLS; subsequent PTT further reduces tumor recurrence and extends animal survival significantly.

### Dosing Studies

The dose used in preliminary studies as well as a lower and higher dose will be utilized to understand the impact of dosing on tumor uptake. Five Balb/c mice per group will be injected in a mammary breast fat pad with 4T-Luc tumors. When tumors reach approximately 100 mm³ animals will undergo baseline imaging and then be IV injected with AKRO-6qc. As in FIG. 3, the level of tumor uptake will be determined measuring total radiant efficiency with the IVIS imaging system.

### pK Analysis

Once dose and time to maximum tumor accumulation are optimized, the optimal dose will be utilized to measure pharmacokinetics. 5 mice will be injected with AKRO-6qc and then 50 μl blood will be withdrawn at 5, 10, 15, 60, minutes and then at 4, 12, 24, and 48 hours. Blood will be used to generate plasma and the amount of AKRO-6qc in blood plasma will be determined by LCMS (Shimadzu HPLC Series 40 and LCMS-2050). Non-plasma will be extracted for identification of probe content as well. Mice bearing tumor will also be assessed for pK to determine if tumors affect the measurements.

Biodistribution and histology: Five control mice and 5 mice bearing 100 mm³ tumors will be injected with the optimal dose of probe and at the time measured for maximum tumor accumulation the animals will be sacrificed and tumors and organs (set 1) or organs (set 2) will be removed, weighed and quantified using fluorescence measurements using the IVIS system. For larger organs only a portion of the organ will be used. Uptake will be expressed as fluorescence/gram of tissue. Organs will include: liver, spleen, kidneys, lungs, heart, brain, and tumors (where applicable).

### Detection and Optimization of PTT Effects In Vivo

PTT Detection and tissue destruction. Six to eight-week-old female Balb/c immunocompetent mice (female mice are best to support breast cancer growth) will be implanted orthotopically with 5,000-10,000 4T1-Luc cells in lower abdominal breast fat pads. Five mice will be inoculated with 4T1-Luc tumors and when tumors reach 100 mm³, the optimal dose of AKRO-6qc administered. Five AKRO-6qc injected mice will serve as non-irradiated controls. Following the optimal time after injection the tumors will be irradiated and surface temperature of the tumor immediately monitored prior to imaging. To assess thermal changes in tumors immediately after irradiation we will utilize a near-infrared FLIR One Pro LT Pro-Grade photothermal camera system (FLIR One Pro) designed to measure temperature changes with 0.1-degree sensitivity and 4,800 pixel IR image resolution. Two hours after irradiation the tumors will be extracted and will undergo histological analysis wherein tumors will be paraformaldehyde fixed and embedded into paraffin blocks. H&E staining will be carried out to look at general cell morphology and necrosis. Immunmohistological fluorescence microscopy will be utilized to identify macrophage population, as described above, and to determine the extent of apoptosis. Heterogeneity of these effects throughout the tumor will also be assessed, including necrosis. Surrounding normal tissues will also be assessed for collateral damage. Finally, 3 groups of animals (5 each) will be utilized to measure the impact of different radiances of light on tumor destruction.

Evaluation of the Antitumor Activity of AKRO 6qc-ICG In Vivo Using Light Irradiation Alone

In this study, we will test the effect of AKRO-6qc in immunocompetent mice bearing 4T1-Luc tumors (as described above, other BCa tumor models may also be tested). Animals will be observed every other day with caliper measurements and luciferase imaging until tumors reached about 100 mm³ (tumor volume-Length X width²/2). Mice will then receive the optimal dose of AKRO-6qc through tail vein injection, determined above. At optimal uptake (likely 24-26 hours) irradiated with 808 nm light for the optimal time for PTT activation, determined above. Animals will be imaged before and after light optical light irradiation, determined above. Mice will be weighed, and tumor size will be measured every other day for 80 days according to CWRU approved IACUC protocol. Caliper measurements will be used for every other day measurements, but once a week luciferase imaging will be utilized to monitor metastatic disease. Animals will be divided into 4 groups: (1) mice receiving PBS; (2) mice receiving AKRO-6qc with irradiation; (3) mice receiving AKRO-6qc, but not receiving irradiation; and (4) mice receiving PBS and irradiation. Each group will have 10 mice based on previous studies and power calculations to get significant results. When tumors become too large or the animals are moribund they will be euthanized. Data will be reported as body weight over time, tumor size over time, and Kaplan-Meier survival plots. On day 80, animals will be sacrificed, and organs collected for histological analysis to examine if there is any damage to the organs. Moribund animals will be studied earlier. While we attempt to determine if the AKRO-6qc probe can be used as a PTT agent it is known that 4T1 tumors are highly metastatic. Therefore, using luciferase and fluorescence imaging of AKRO-6qc we will also assess metastasis non-invasively. If lung metastasis is detected, we will compare mice with and without treatment to determine if irradiation of the primary tumor impacts metastatic migration to the lungs and possibly liver. Since the luciferin enzyme is a “non-self” protein, we will also choose the best conditions for response and utilize 4T1 cells that do not express LUC to determine if tumor attenuation depends on expression of LUC.

Evaluation of the Antitumor Activity of AKRO 6gc-ICG In Vivo Implementing FIGS+PTT

Mice will be implanted with 4T1-Luc tumors as described above and then undergo surgery followed by light irradiation. Ten mice for each test set will be utilized to achieve statistical significance as described in. Mice will be randomly divided into three groups: white light surgery (WLS), fluorescence guided surgery (FIGS), and FIGS, followed by PTT (FIGS+PTT). All mice will be dosed with AKRO-6qc at the determined optimal time using the determined optimal dose, followed by surgery. WLS will be performed under room light. For FIGS, tumors will be removed under the guidance of real-time fluorescent camera imaging (Curadel RP1 camera) and the wounds will be sutured for both WLS and FIGS groups. For the FIGS+PDT group of mice, the resection bed will be directly irradiated with 808-nm laser using an optical fiber diffuser (Modulight 7710 Laser system). Initially, optimal light irradiation will be used, determined above, but temperature achieved in preliminary studies will be monitored and achieved under control of a thermal camera. After PTT, the wounds will be sutured. Tumor re-growth, if any, will be monitored using both bioluminescent and fluorescent signals from the wounds for 80 days.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of treating cancer in a subject in need thereof, the method comprising:
administering to the subject a cancer cell-targeted cyanine near-infrared fluorophore;
detecting the administered cancer cell-targeted cyanine near-infrared fluorophore in the subject to determine the location and/or distribution of the cancer cells in the subject;
surgically resecting cancer cells detected using the administered cancer cell-targeted cyanine near-infrared fluorophore at the determined location and/or distribution in the subject; and
irradiating remaining or residual cancer cells at the determined location after surgical resection that are detected by, bound to, and/or complexed with the cancer cell-targeted cyanine near-infrared fluorophore at a wavelength effective to ablate the remaining or residual cancer cells.

2. The method of claim 1, wherein the cancer cell-targeted cyanine near-infrared fluorophore is a cancer cell-targeted heptamethine cyanine near-infrared fluorophore.

3. The method of claim 1, wherein the cyanine near-infrared fluorophore includes indocyanine green (ICG) or an analogue thereof.

4. The method of claim 3, wherein the cyanine near-infrared fluorophore is linked to at least one cancer cell targeting moiety.

5. The method of claim 4, wherein the cancer cell targeting moiety binds to, complexes with, and/or is cleaved by a cancer cell molecule.

6. The method of claim 5, wherein the cancer cell molecule and/or molecule in the cancer cell microenvironment includes at least one of cancer cell surface antigen and/or enzyme that is overexpressed the cancer cell and/or in the cancer cell microenvironment.

7. The method of claim 6, wherein the cancer cell antigen comprises at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, cathepsin, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

8. The method of claim 7, wherein targeting moiety comprises at least one of a peptide, protein, nucleic acid, or small molecule that targets the cancer cell antigen.

9. The method of claim 8, wherein cell-targeted heptamethine cyanine near-infrared fluorophore has the formula: 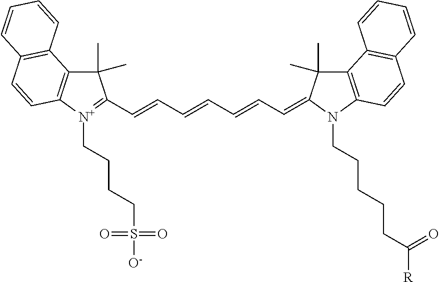
or a pharmaceutically acceptable salt thereof;
wherein R includes the targeting moiety and optional spacer.

10. The method of claim 6, wherein the enzyme that is overexpressed in the cancer cell microenvironment comprises cathepsin.

11. The method of claim 10, wherein the cell-targeted heptamethine cyanine near-infrared fluorophore comprises at least one of AKRO-6qc (6qc-ICG) or VGT-309.

12. The method of claim 1, wherein the cancer is selected from colorectal cancer, breast cancer, lung cancer, melanoma, hepatoma, head and neck cancers, glioma, squamous cell carcinomas of the lung, ovarian cancer, uterine cancer, prostate cancer, gastric carcinoma, cervical cancer, esophageal carcinoma, bladder cancer, prostate cancer, kidney cancer, brain cancer, bone cancer, pancreatic cancer, skin cancer, cutaneous or intraocular malignant melanoma, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemias solid tumors of childhood, lymphocytic lymphoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, medulloblastoma pilomatrixomas, endometrial cancer, multiple myeloma, or T-cell lymphoma.

13. The method of claim 1, wherein the cancer cells comprise at least one of a glioma, lung cancer, melanoma, breast cancer, or prostate cancer cell.

14. The method of claim 1, wherein the cancer cell-targeted cyanine near-infrared fluorophore is administered systemically, locally, or topically to the subject.

15. The method of claim 1, wherein the wavelength effective to ablate the remaining or residual cancer cells is a wavelength effective for photodynamic therapy or photothermal therapy.

16. The method of claim 1, further comprising administering to the subject an immune checkpoint inhibitor.

17. The method of claim 16, wherein the immune checkpoint inhibitor is an inhibitor for any of immune checkpoint molecules selected from the group consisting of PD-1, CTLA-4, TIM-3, BTLA, LAG-3, A2aR, KIR, VISTA, TIGIT, PD-L1 PD-L2, CD80, CD86, GAL-9, HVEM, CD160, MHC class II, B7-H3, B7-H4, B7-H5. B7-H6, and B7-H7, or a combination of two or more inhibitors thereof.

18. The method of claim 16, wherein the immune checkpoint inhibitor is selected from an antibody against the immune checkpoint molecule, an antigen-binding fragment of the antibody, or a combination thereof.

19. The method of claim 16, wherein the immune checkpoint inhibitor is selected from the group consisting of nivolumab, pembrolizumab, cemiplimab, avelumab, atezolizumab, and durvalumab.
